(19) United States
(12) Patent Application Publication
(10) Pub. No.: US 2009/0171436 A1
(43) Pub. Date: Jul. 2, 2009

(54) GRAFTS AND STENT GRAFTS HAVING A RADIOPAQUE BEADING

(76) Inventors: R. Michael Casanova, Scottsdale, AZ (US); P. Pathak, Phoenix, AZ (US)

Correspondence Address: PROSKAUER ROSE LLP, 1001 PENNSYLVANIA AVE, N.W.,, SUITE 400 SOUTH, WASHINGTON, DC, 20004, US

(21) Appl. No.: 12/092,561

(22) PCT Filed: Nov. 9, 2006
(86) PCT No.: PCT/US2006/060704

§ 371 Date: Sep. 17, 2008

Related U.S. Application Data
(60) Provisional application No. 60/734,726, filed on Nov. 9, 2005.

(51) Int. Cl. A61F 2/06; A61B 5/05

(52) U.S. Cl. 623/1.13; 623/1.34; 600/424

(57) ABSTRACT

A device and method provides a graft having a layer of synthetic non-metallic material including a first surface and a second surface spaced apart from the first surface. The device further includes a beading coupled to the layer and a radiopaque agent coupled to the beading. Another device and method provides a implantable prosthesis having a stent frame, a first inner layer and a second outer layer defining a central axis. The implantable prosthesis further includes a beading coupled to at least one the layers.

### PRIORITY DATA AND INCORPORATION BY REFERENCES

This application claims benefit of priority to U.S. Provisional Patent Application No. 60/734,726 filed Nov. 9, 2005 which is incorporated by reference in its entirety.

### TECHNICAL FIELD

The present invention relates generally to medical devices, and more particularly to a radiopaque beading for implantable devices.

### BACKGROUND OF THE INVENTION

Unless mentioned specifically, the term radio-opaque and radiopaque have the same meaning. Artificial grafts, stent grafts and related endoluminal devices are currently used by operators to treat tubular body vessels or ducts that become so narrowed (stenosed) that flow of blood or other biological fluids is restricted. Such narrowing (stenosis) occurs, for example, as a result of the disease process known as arteriosclerosis. These products can be used to “prop open” blood vessels, they can also be used to reinforce collapsed or narrowed tubular structures in the respiratory system, the reproductive system, bile or liver ducts or any other tubular body structure. Vascular grafts made of polytetrafluoroethylene (PTFE) are typically used to replace or repair damaged or occluded blood vessels within the body. However, they may require additional means for anchoring the graft within the blood vessel, such as sutures, clamps, or similarly functioning elements to overcome retraction.

PTFE has proven unusually advantageous as a material from which to fabricate blood vessel grafts or other implantable prostheses, because PTFE is extremely biocompatible, causing little or no immunogenic reaction when placed within the human body. In its preferred form, expanded PTFE (ePTFE), the material is light, porous and readily colonized by living cells so that it becomes a permanent part of the body. The process of making ePTFE of vascular graft grade is well known to one of ordinary skill in the art. Suffice it to say that the critical step in this process is the expansion of PTFE into ePTFE. This expansion represents a controlled longitudinal stretching in which the PTFE is stretched to several hundred percent of its original length. Examples of ePTFE grafts are shown and described in U.S. Pat. Nos. 5,641,443; 5,827,327; 5,861,026; 5,641,443; 5,827,327; 6,203,735; 6,221,101; 6,436,135; and 6,589,278, each of which is incorporated in its entirety by reference. Grafts made from materials other than ePTFE include, for example, Dacron mesh reinforced umbilical tissues, bovine collagen, polyester knitted collagen, tricot knitted polyester collagen impregnated, and polyurethane (available under the trademark “Vectra®”) have been utilized.

Implantation of a graft into the vasculature of a patient involves very precise techniques. Generally, the device is guided to the diseased or damaged portion of a blood vessel via an implantation apparatus that deploys the graft at the desired location. In order to pinpoint the location during deployment, the operator will generally utilize a fluoroscope to observe the deployment by means of X-ray. In addition, visualization of the implanted device is essential for implantation, follow-up inspection and treatment. Accordingly, in order to implant the graft or implantation device by fluoroscopy, some portion of the device should preferably be radiopaque.

A graft can be generally delivered to the damaged or diseased site via a constraining member in the form of a catheter or sheath and can be deployed by removing the constraining member. In order to direct the device or graft to the precise location for deployment, the radiopacity is preferably incorporated into the device or the constraining member to confirm the correct placement within the vessel. A problem can arise in delivering a graft via a sheath. In particular, if there is any interference between the graft and the sheath, the delivery procedure is complicated by requiring additional manipulation of the graft to migrate through the sheath and to the site of the stenosis.

In addition to visually verifying location of the implanted graft, it may be necessary to visually verify the orientation of the graft, and/or visually determine if the implant has been mislocated, for example, twisted or kinked. Generally, the wall thickness of a graft is relatively thin ranging from about 50 microns to about 1000 microns. The thin wall and dimensions of the implant device provides flexibility to the implant which assists in manipulation of the implant around tissue during implantation. Use of a thin wall graft can permit the manufacture of smaller devices which could be delivered using small size catheter based delivery system. It is believed however that, that these thin wall devices may be subject to structural degradation such as, kinking, during implantation.

Stents have been used in combination with vascular grafts, i.e. “stent grafts,” to provide endovascular prostheses which are capable of maintaining their fit against blood vessel walls. The use of grafts along with stents also serves to overcome a problem found with stents where smooth muscle cells and other tissues can grow through the stent's mesh-like openings, resulting in restenosis of the vessel. Stent grafts are a prosthetic device designed to maintain the patency of various vessels in the body, including the tracheobronchial tree. The device can include a balloon expandable stent encapsulated within ePTFE or alternatively a self-expanding Nitinol stent encapsulated within ePTFE and pre-loaded on a flexible delivery system. One example of the latter is known commercially as “Fluency®,” which is marketed by C.R. Bard Peripheral Vascular Inc. Examples of such stent grafts is shown and described in U.S. Pat. Nos. 6,053,941; 6,124,523; 6,383,214; 6,451,047; and 6,797,217, each of which is incorporated in its entirety by reference. The field of covering stents with polymeric coatings and ePTFE in particular has been substantially explored by those skilled in the art. One popular way of covering the stent with ePTFE material is to encapsulate it within two layers of ePTFE, which are subsequently fused together by heat in places where the two layers are in contact through openings in the stent wall. This provides a solid one-piece device that can be expanded and contracted without an ePTFE layer delaminating.

Implantation of an encapsulated stent into the vasculature of a patient involves very precise techniques. Generally, the device is guided to the diseased or damaged portion of a blood vessel via an implantation apparatus that deploys the encapsulated stent at the desired location. In order to pinpoint the location during deployment the operator will generally utilize a fluoroscope to observe the depolyment by means of X-rays. Deployment of an encapsulated stent at an unintended location can result in immediate trauma, as well as increasing the invasiveness associated with multiple deployment attempts and/or relocation of a deployed device. In addition, visualization of the implanted device is essential for implantation, follow-up inspection and treatment. Accordingly, in order to implant the encapsulated stent using fluoroscopy, some portion of the stent or implantation device should be radiopaque.

Stents that are implanted and expanded within a blood vessel using a balloon catheter can be located by fluoroscopy because the balloon catheter can have radiopaque features incorporated therein that may be used as a visual marker. However, if the balloon moves after expansion of the stent, correct placement of the stent, in the absence of a radiopaque marker incorporated into the stent, cannot be confirmed. A self-expanding stent can be generally delivered to the damaged or diseased site via a constraining member in the form of a catheter or sheath and can be deployed by removing the constraining member. In order to direct the device to the self-expanding stent to the precise location for deployment, the radiopacity can be incorporated into the device or the constraining member to facilitate the correct placement within the vessel.

### DISCLOSURE OF INVENTION

A preferred embodiment provides a graft device with a layer of synthetic non-metallic material having a first surface and a second surface spaced apart from the first surface. The graft device further includes a beading coupled to the layer and a radiopaque agent coupled to the beading. Preferably, the beading provides kink resistance, and the coupling of the radiopaque agent to the beading provides a radiopaque beading. Preferably, the layer of synthetic non-metallic material forms an elongated substantially tubular member. The second surface preferably forms the outer surface of the tubular member, and the radiopaque beading is further preferably spirally wrapped about the outer surface. In addition, the radiopaque beading preferably defines a substantially rectangular cross-sectional area. In one embodiment, the radiopaque beading includes a radiopaque material embedded in a polyurethane material. In yet another embodiment, the radiopaque beading includes a radiopaque core disposed within a polytetrafluoroethylene shell. Preferably, the radiopaque material includes 20% by weight of Barium Sulfate. Alternatively, the radiopaque beading is formed from a paste having about 20% tantalum powder. Yet further in the alternative, the radiopaque beading is formed from a paste having about 20% to about 40% Barium Sulfate. More preferably, the radiopaque beading is a tape of 40% tantalum powder and 60% PTFE.

Another embodiment provides a method of forming a graft device which preferably includes disposing a radiopaque agent in a polymeric shell, compressing the radiopaque agent and shell to form a billet, extruding the billet so as to form a radiopaque beading; and wrapping the beading about a graft material so as to define a graft device. The method further provides that the wrapping includes the beading about the graft. The method further preferably includes applying a solvent.

In yet another embodiment according to the present invention, a stent graft device includes a stent frame having a first inner layer and a second outer layer disposed about a central axis. The stent graft further includes a beading coupled to at least one of the layers. In addition, the stent graft device can further include a radiopaque agent coupled to the beading. The coupling of the radiopaque agent to the beading provides a radiopaque beading.

In yet another preferred embodiment, provided is a method of forming a stent graft device. The stent graft device is formed, at least by, including disposing a radiopaque agent in a polymeric shell, compressing the radiopaque agent and shell to form a billet, extruding the billet so as to form a radiopaque beading; and wrapping the beading about a graft material so as to define a graft device.

A kink in a graft device can substantially reduce blood flow therethrough and make the graft essentially useless. Thus, the ability to resist kink during and after surgical implantation can be a factor in restoring blood flow. Generally, in commercial vascular graft products such as CENTERFLEX® graft, for example, beading is provided to resist kinking in the graft. In a preferred embodiment according to the present invention, beading provides radio-opacity as well as kink resistance.

Another preferred embodiment provides a method of observing a position of a implantable prosthesis in a body. The method preferably includes disposing a implantable prosthesis having a radiopaque beading in the body and exposing the body to an electromagnetic energy. The method further preferably includes fluoroscopically observing at least a portion of the beading to determine the position of the implantable prosthesis in the body.

Accordingly, a properly configured radiopaque beading can facilitate meeting the visual needs of an operator in addition to providing structural rigidity to an implant device. More specifically, a radiopaque beading coupled to a graft or stent graft device can provide the necessary visual cues to assist in the implantation, follow-up and treatment of the device. The radiopaque beading can also be configured to reduce kinking in a graft by providing sufficient structural support to the implant without significantly reducing flexibility. Moreover, the use of the radiopaque beading can be preferably configured to minimize line contact between a graft and a delivery sheath or between a stent graft and a delivery sheath by limiting contact to line contact in the area defined between the radiopaque beading and the sheath. It is believed that minimizing surface contact or interference between the stent and the sheath can minimize the force required to withdraw the sheath covering the self-expanding stent.

### BRIEF DESCRIPTION OF THE DRAWINGS

The accompanying drawings, which are incorporated herein and constitute part of this specification, illustrate exemplary embodiments of the invention, and together, with the general description given above and the detailed description given below, serve to explain the features of the invention. It should be understood that the preferred embodiments are some examples of the invention as provided by the appended claims.

FIG. 1 illustrates a preferred graft device.

FIG. 1A is an X-ray view of the graft device of FIG. 1.

FIG. 2 is a cross-sectional view of a first embodiment of a radiopaque beading used in the device of FIG. 1.

FIG. 3 is a cross-sectional view of another embodiment of a radiopaque beading.

FIG. 4 is an illustrative embodiment of a preform barrel.

FIG. 5 is a cross-sectional view of another embodiment of a preferred graft device.

FIG. 6 illustrates another embodiment of a preferred graft device.

FIG. 7 illustrates yet another embodiment of a preferred graft device.

FIG. 8 illustrates a preferred stent graft with radiopaque beading.

FIG. 9 is an X-ray view of the stent graft with radiopaque beading of FIG. 8.

FIG. 9A is a cross-sectional view of the radiopaque beading of FIG. 8.

FIG. 10 is a cross-sectional view of yet another radiopaque beading.

FIGS. 11, 11A, and 11B are various perspective and cross-sectional views of another stent graft having a radiopaque beading.

FIG. 12 is an illustrative fluoroscopic image of a stent graft having a beading formed by a combination of polyurethane and a radiopaque agent.

### MODE(S) FOR CARRYING OUT THE INVENTION

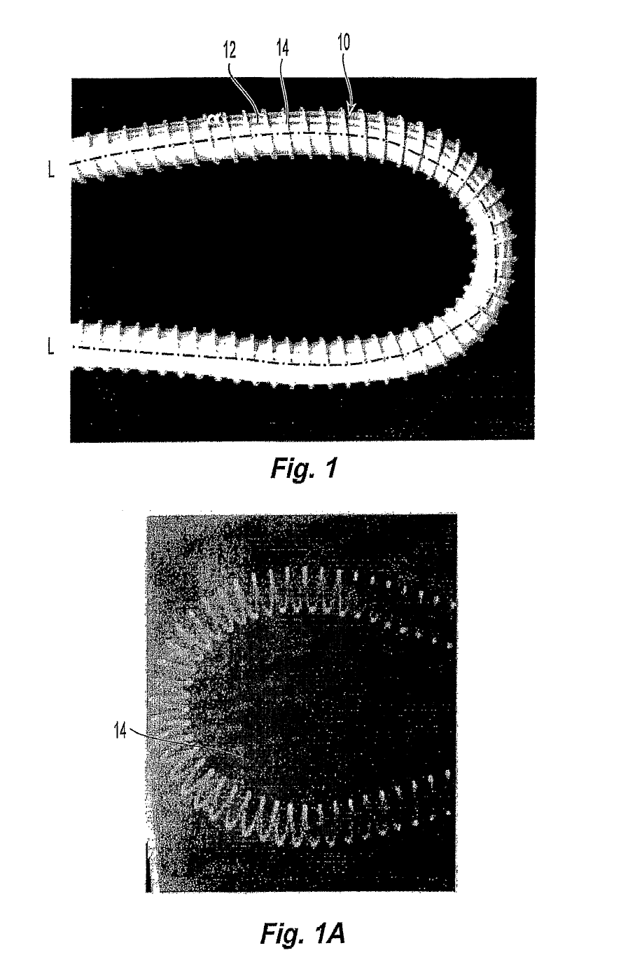

FIG. 1 shows a preferred embodiment of a medical device implant 10 having an outer surface 12 and an inner surface (not shown). The device 10 is preferably a graft device and its outer surface 12 preferably defines a substantially tubular member about a central axis L-L of the device 10. Preferably, the device 10 defines a substantially circular cross-section perpendicular to the central axis, although other cross-sectional geometries are possible such as, for example, rectangular or oval. The device 10 is preferably configured for migration through a blood vessel to engage, for example, a stenosis. Alternatively, the device 10 can be substantially spherical or any other geometry appropriately dimensioned for implantation and migration in blood vessels or other tissue. Exemplary graft devices 10 include IMPRA CARBOFLO® and CENTERFLEX® by Bard Peripheral Vascular, Inc., Tempe, Ariz.

Disposed or coupled to the outer surface is a beading 14. “Beading” as used herein means a substantially solid segment, rod, wire or elongated structure capable of being shaped into various cross-sectional configurations. Preferably coupled to the beading 14 is a radiopaque agent to provide a visual indicator to an operator viewing the device 10 under fluoroscopic observation, as seen for example in FIG. 1A. More specifically, the beading with radiopaque agent, i.e. the radiopaque beading 14, provides an operator with a visual indicator to determine or verify the location and/or orientation of the device 10 upon implantation in a blood vessel or other tissue. The radiopaque beading 14 is preferably wound about the outer surface 12 so as to substantially circumscribe the central axis of the device 10. Alternatively, the radiopaque beading can be disposed on the outer surface 12 so as to be substantially to one side of the central axis. The radiopaque beading further preferably forms a continuous wrapping about the central axis of the device 10 so as to form a continuous contour line on the outer surface of the device 10. Alternatively, the radiopaque beading 14 can be formed by a series of segments aligned about the outer surface 12. Further in the alternative, the radiopaque beading 14 can be formed by a plurality of individual rings dimensioned and configured to be disposed about the device 10 and spaced apart along the central axis. Each of the plurality of rings can define its own geometric shape, for example, a ring of beading may be substantially rectangular or circular so long as the ring defines a sufficient interstitial space to be disposed about the device 10. Preferably, the radio-opaque beading 14 is helically wrapped about the outer surface 12 so as to provide a desired level structural rigidity, for example, kink resistance along the length of the central axis of the device 10. The helical wrapping of the radiopaque beading 14 can maximize coverage of the outer surface 12 while minimizing the overall surface area of the beading 14. In addition, the preferred continuous helical wrapping beading 14 provides contour lines that provide additional visual cues to the user during and after implantation. For example, an untwisted implanted device 10 with preferred radiopaque beading 14 optimally appears as a series of parallel lines along the central axis of the device 10. Conversely, any twisting or bending in the device 10 would appear as converging lines in the radiopaque beading 14. Other coverage configurations for radiopaque beading 14 can be employed such as, for example, forming distinct circular radiopaque beading about the outer surface 12 along axial length of the device 10. The circular radiopaque beading 14 can be substantially perpendicular to the central axis or alternatively be oblique to the central axis. In another alternative coverage configuration, the beading 14 can be elongated strips of radiopaque beading radially spaced about the central axis of the device 10.

The device 10 can be a tubular member made from a graft material which can be a non-metallic material. Preferably, the graft material is expanded polytetrafluoroethylene (ePTFE), but alternative non-metallic materials are possible for forming the device 10 such as, for example, Dacron, polyester, polytetrafluoroethylene (PTFE), ePTFE, polyurethane, polyurethane-urea, siloxane, and combinations thereof. The material can include additional additives such as, for example, bio-active agents.

To form the device 10, the non-metallic material is preferably formulated into a resin or paste which is then compressed within a cylinder to form billet of the material, for example, an ePTFE billet. Resins of different materials can be also be combined to form a resin composite having various desired properties, for example, the ePTFE resin can be combined with hydroxyapatite (HA) to produce a material having increased biocompatibility and bioactivity. The billet is then preferably extruded and cured to form the tabular member 10.

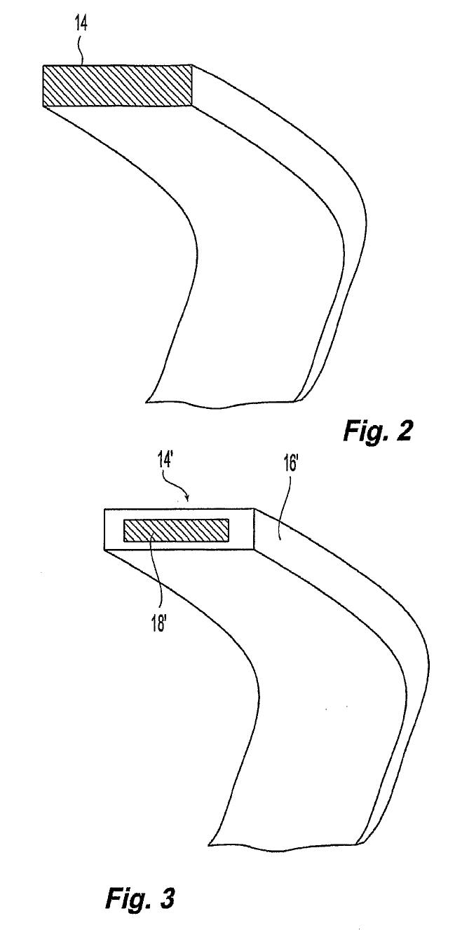

Disposed about the device 10 in spiral configuration is the radiopaque beading 14. Shown in FIG. 2 is a cross-sectional view of one embodiment of the radiopaque beading 14. The radiopaque beading 14 is preferably rectangular in cross-section to provide the maximum contact surface for coupling to the device 10. Alternatively, the beading 14 can be any other geometry in cross-section such as, for example, circular, oval or polygonal. The preferred cross-sectional area of the beading 14 is dimensional so as to have a length ranging from about 1 millimeter to about 2 millimeters and a width ranging from about 100 microns to about 500 microns. More preferably the cross-sectional area of the beading 14 is dimensioned so as to have a length of about 1 millimeter and a width of about 500 microns. Preferably, the elongated side of the beading 14, in cross-section, forms the interface between the radiopaque beading 14 and the device 10. In another preferred embodiment, the beading 14 is substantially circular in cross-section, and the diameter of the beading 14 is preferably about 0.67 millimeters.

The radiopaque beading 14 is preferably made of a biocompatible polyurethane material such as, for example, Carbothane® PC-3575 by Noveon, Inc. (Thermedics Division) Cleveland, Ohio with a Barium Sulfate salt embedded in the polyurethane as a radiopaque agent. The Carbothane preferably has a 72 Shore D hardness and the Barium Sulfate is present at 20% by weight. Generally, a concentration of Barium Sulfate greater than 10% is sufficient to provide radiopacity. Preferably, the concentration Barium Sulfate in the beading 14 ranges from about 20% to about 40% to provide the radiopacity. Alternatively, the radiopaque beading can be made from other biocompatible polymers such as, for example, Dacron, polyester, PTFE, ePTFE, polycarbonates, polysulfone, polyethylene, polypropylene, polyurethane-urea, siloxane, and combinations thereof. In addition, other materials can serve as the radiopaque agents such as, for example, tantalum, tungsten, gold, silver or other metallic powders or salts such as calcium or HA salt.

The radiopaque beading 14 is preferably formed by extrusion. In one embodiment, the Carbothane PC-3575 material and 20% by Barium Sulfate are combined in a composite resin or paste in which the Barium Sulfate is preferably dispersed throughout the polyurethane material. The composite paste is preferably loaded in a press device to compress the material into a billet. The billet is then preferably extruded to form the radiopaque polyurethane beading 14.

The polyurethane radiopaque beading 14 can be coupled to the device 10 to produce the implantable graft with radiopaque marker shown in FIG. 1. In a preferred method of coupling the beading 14 to the outer surface 12 of the device 10, the beading 14 is preloaded onto the outer surface 12. More specifically, the beading 14 is placed under tension, preferably about 500 grams of force, and then the beading 14 is wound through a solution of solvent about the outer surface 12 of the graft which can be temporarily mounted to a mandrel. Preferably, the spacing between adjacent windings of the beading 14 is about 1 millimeter to about 2 millimeters. As previously noted, the elongated side forming the rectangular cross-sectional area of the beading is engaged or coupled to the outer surface 12. The solution of solvent can dissolve polyurethane, and therefore when applied to the beading 14 in the wrapping process, the solvent can form a mechanical bond between the beading 14 and the outer surface 12. Preferably, the solvent is tetrahydrofuran (THF), but other aprotic solvents can be used. The solvent is preferably applied by any suitable technique such as, spraying or coating and preferably by pulling the beading through a solvent bath. Thereafter, the solvent can be subsequently removed by preferably post-curing the assembled device 10 and beading 14.

FIG. 1A shows a fluoroscopic or X-ray view of the device 10 with radiopaque polyurethane beading 14. The radiopacity of the beading 14 is manifested in the imaging of the head 14 contrasted with the radiolucent outer surface 12 of the device 10. Consequently, as long as an ordinary observer can determine that the lines provided by the radiopaque beading 14 in a fluoroscopic display medium has a darker or higher contrast image than the remainder of the device 10, then the radiopacity of the beading 14 would be deemed to be greater than a minimum level needed for the beading to function as a radiopaque marker in a mammalian body. Alternatively, a machine vision with the ability to recognize discrete levels of contrast can be utilized to provide an objective indicator of the effectiveness of the radiopacity of the radiopaque beading 14.

The beading 14 is preferably mounted or coupled to the device 10 by winding the polymeric beading under tension on the surface 12 of the device 10. The beaded graft assembly can then be sprayed with a solvent such as, for example, tetrahydrofuran in an amount sufficient solvent to adhere the beading to the surface but not dissolving the beading. Alternatively, beaded graft assembly 14, 10 can be dipped in the solvent such as tetrahydrofuran for five seconds to 300 seconds, more preferably in the range of thirty to sixty seconds. The beaded graft assembly is removed from the solvent and the solvent is preferably evaporated by air drying. The beaded graft assembly is preferably dried in oven at 70° C. for twelve hours to remove the solvent completely. The short dipping time is preferably designed to bond the beading to graft surface without dissolving the beading completely. Other solvents such as acetone, dimethyl acetamide, dimethyl sulfoxide, n-methyl pyrrolidinone, dioxane may also be alternatively used. Solvents that evaporate rapidly are most preferred, and solvents with boiling point below 70° C. are furthermore preferred. In certain application, it is preferable to provide a beading that can be peeled during surgical implantation. The solvent bonding methods described above can provide a removable beading that can be easily peeled away. More specifically, the solvent bonding method can facilitate manual separation of the beading and the graft material upon application of an appropriate force. However, the bead peeling can occur without substantially damaging the graft surface. Again more specifically, the bead peeling can progress so as to separate a portion of the beading from the graft material without disturbing the bond between the graft material and the remainder of the beading.

Another preferred embodiment of the radiopaque beading is shown in FIG. 3 in which the radiopaque beading 14′ includes an outer lumenal layer of non-radiopaque material 16′ surrounding a radioque core 18′. The outer layer 16′ is preferably ePTFE so as to provide an ePTFE beading 14′ with desired peeling properties as is provided in known beaded products such as, for example, CENTERFLEX® graft by Bard Peripheral Vascular, Tempe, Ariz. Alternatively, other polymeric materials can be used to form a shell to which the radiopaque agent can be coupled to or disposed within. Such as polymeric materials include, for example, Dacron, polyester, polyurethane, PTFE, polycarbonates, polysulfone, polyethylene, polypropylene, polyurethane-urea, siloxane, and combinations thereof. The radiopaque core 18′ is preferably 20% by weight of Barium Sulfate salt material. Alternatively, the radiopaque core 18′ can be made from other radiopaque agents including tantalum, tungsten, gold, silver or other metallic powders or salts such as calcium or hydroxyapatite (HA) salt.

Although the ePTFE beading 14′ can be made by a variety of suitable techniques, a preferred technique is described as follows. A compounding of a polymeric compound is generated by sifting PTFE resin with a suitable amount of lubricant such as, for example, Isopar H, at 30% by weight of the PTFE to enable the PTFE to flow through extrusion equipment. The combined PTFE resin and lubricant are then placed in a shaker device and shaken so that the lubricant coats and penetrates each of the PTFE resin particles. The thoroughly mixed combination of PTFE resin and lubricant is then incubated in a warming cabinet overnight which is maintained at a temperature of approximately 85 degrees Fahrenheit (85° F.). The incubation period is believed to allow for a further and more equal dispersion of the lubricant throughout the PTFE resin.

If desired, the PTFE resin can be further mixed and heated with other suitable bio-active material as part of an optional compounding process. For example, the PTFE resin can be compounded with a suitable hydroxyapatite (HA) material to produce a beading material for increased biocompatibility and bioactivity in order to, for example, promote endothelial cell growth for the reduction of intimal hyperplasia.

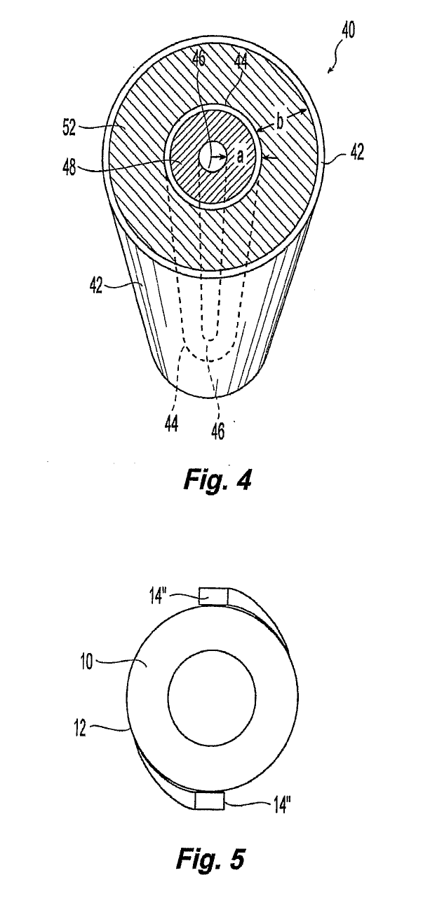

The PTFE resin or its compound can be preformed into a compressed cylinder by series of process steps. First the resin can he poured into an inner barrel of a preformer by directing it through a funnel which is fit to the outside of the inner barrel. FIG. 4 illustrates a preferred embodiment of a divided preform barrel 40 which can be used in preforming a resin into a compressed cylinder. The divided preform barrel 40 preferably includes an outer hollow cylindrical member 42, an optional inner hollow cylindrical member 44, and a central solid cylindrical member 46. The inner hollow cylindrical member 44 can be concentrically contained within the outer hollow cylindrical member 42. Details of a similar process are shown and described in U.S. Pat. Nos. 5,827,327; 5,641,443; and 6,190,590, each of which is incorporated in its entirety by reference.

The PTFE resin can be poured within a first area 52 located between the outer hollow cylindrical member 42 and a solid cylindrical member 46. The first area 52 can be divided by one or more inner members 44 to define a secondary area 48 for receipt of a radiopaque material such as, for example, a 20% by weight Barium Sulfate compound to form the radiopaque core 18′.

In one of the preferred embodiments, the outer hollow cylindrical member 42 has a radius greater than the radius of the inner hollow cylindrical member 44. The diameter of the components which form the preform barrel 40 will vary depending on the size and type of graft that is being produced. A preferred embodiment of the preform barrel 40 can have a radius of approximately 1.5 inches. The secondary area 48 between the inner hollow cylindrical member 44 and the central solid cylindrical member 46 can have a radius of approximately 0.38 inches, the inner hollow cylindrical member 44 can have a wall thickness of approximately 0.07 inches, and the first area 52 located between the outer hollow cylindrical member 42 and the inner hollow cylindrical member 44 can have a radius of approximately 0.6 inches.

Alternatively, a radiopaque paste or resin can be partially or fully embedded in a portion of the inner surface of the PTFE resin without the use of an inner divider member 44. The radiopaque paste can be formed from a tantalum powder. For example, the radiopaque paste can be formed from a sixty percent (60%) tantalum paste combined with an ePTFE paste. Additionally, other suitable materials can be utilized to form the radiopaque paste, for example, gold or titanium. Further in the alternative, the radiopaque paste can be formed from a Barium Sulfate mixture. For example, the radiopaque paste can be include an ePTFE paste mixed with twenty to forty percent (20-40%) Barium Sulfate. In a preferred embodiment, the radiopaque paste is formed into an elongated strip that can be disposed along the length of the inner surface of the PTFE resin. Alternatively or in addition to, the radiopaque paste can form a plurality of radiopaque elements that can be aligned along the inner surface of the PTFE resin along its length. The radiopaque paste can be formed into any shape or form. For example, the paste can be formed as sutures, threads and other small pieces such as disks disposed anywhere within the PTFE resin. The continuous or elongated strip of radiopaque material embedded in the inner surface of the PTFE resin can provide the radiopaque core 18′ to the operator viewing the beading 14′ under fluoroscopy.

The assembly of PTFE resin and radiopaque paste markers is preferably compressed to form a billet. The materials are compressed by placing the assembly into the preform barrel 40 on a suitable press such as is shown, for example, in FIG. 3 of U.S. Pat. No. 5,827,327. The press used during the compression of the polymeric compound is driven by a suitable power drive, which forces a top member toward a bottom member to compress the material within the divided preform barrel 40. Hollow cylindrical tubes of varying thickness are used to compress the material within the divided preform barrel 40 by slidably reciprocating around the inner hollow cylindrical member 44, the outer hollow cylindrical member 42, and the center solid cylindrical member 46 of the divided preform barrel 40. After compressing the materials contained within the preform barrel 40, the inner cylindrical member 44 (if used), the outer cylindrical member 42, and the center solid cylindrical member 46 of the divided preform barrel 40 are removed to obtain a compressed cylinder or billet of material. Alternatively, the dividers within the preform barrel may be removed prior to compression, without disturbing the interface between the different compounds, and then compressed to form a billet for extrusion.

The compressed cylinder or billet having an outer PTFE layer and radiopaque core is preferably co-extruded via a suitable device such as, for example, the extruder shown in FIG. 4 of U.S. Pat. No. 5,827,327. Briefly, the compressed cylinder of material is placed within an extrusion barrel. Force is applied to a ram, which in turn expels pressure on the compressed cylinder of material. The pressure causes the compressed cylinder of material to be extruded through extrusion die and issue as a tubular extrudate or beading.

The ePTFE radiopaque beading 14′ can be bonded or coupled to a graft device 10. In a preferred method for bonding the ePTFE radiopaque beading 14′ to a graft device 10, ePTFE beading 14′ can be wrapped about the graft device 10. The graft 10 and beading 14′ can be sintered at a temperature to fuse the beading 14′ with the graft surfaces 12. The sintering temperatures can range from about 340° C. to about 380° C., and preferably from about 355° C. to about 365° C.

FIG. 5 shows a cross-sectional view of another embodiment of a radiopaque beading wrapped about a graft device 10. More specifically, shown in FIG. 5 is a cross-sectional view of a radiopaque beading in the form of a tape 14″. The cross-sectional area of the beading 14″ preferably is rectangular and is further preferably dimensioned such that the tape 14″ is about 2 millimeters wide with a thickness ranging from about 100 microns to about 150 microns. The tape can be formed with a preferred composite resin of about 60% tantalum as a radiopaque agent and 40% PTFE of polymeric material. Alternatively, other polymeric and radiopaque agents can be used. The tantalum and PTFE composite is preferably extruded and expanded three times to form the radiopaque tape 14″. Further in the alternative, an unexpanded tape can be employed. The unexpended tape can provide more radio-opacity as compared to expanded tape presumably due to reduction of density of radio-opaque material. The tape 14″ is preferably bound or coupled to a graft device 10 by wrapping the tape 14″ about a graft device 10 and sintering the assembly to fuse the radiopaque tape 14″ to the device 10. The tape 14″ is preferably bound to the device 10 by sintering the assembly at 340-380° C., preferably at 355° C. to 365° C. for 0.5 to 5 minutes, and preferably for 1-2 minutes.

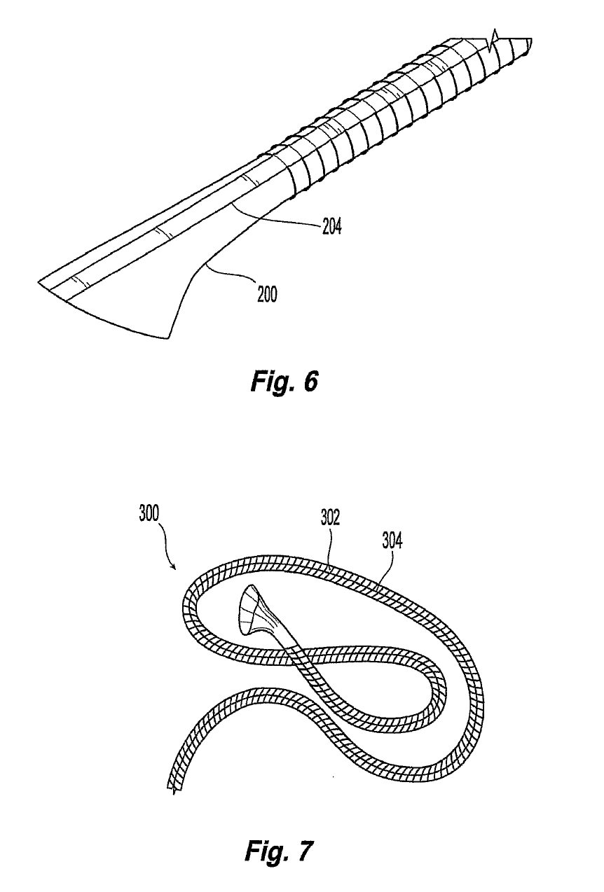

Although the graft device 10 has been described in relation to specific examples noted above, it should be emphasized that variations in the configuration or composition of ePTFE, radiopaque beading, and other design parameters can be utilized with the graft device 10. For example, referring FIGS. 6 and 7, shown are alternative embodiments of a graft, namely vascular bypass grafts 200 and 300. Grafts 200 and 300 can preferably include a helically wound radiopaque beading (not shown) bound to the outer surface. Vascular bypass graft 200 is configured for desired blood flow characteristics for applications above the knees, whereas bypass graft 300 is configured for blood flow characteristics below the knee. Regardless of the structural configurations and applications of the bypass grafts 200 and 300, the grafts 200, 300 can be preferably formed by extruded ePTFE material along with a radiopaque beading 204, 304. That is, a radiopaque beading can be bonded by sintering or solvent bonding to at least one of the luminal and abluminal surfaces of the grafts (200 or 300). Additional examples of various grafts are shown and described in U.S. Pat. Nos. 6,203,735; 6,039,755; 6,790,226, each of which is incorporated in its entirety by reference.

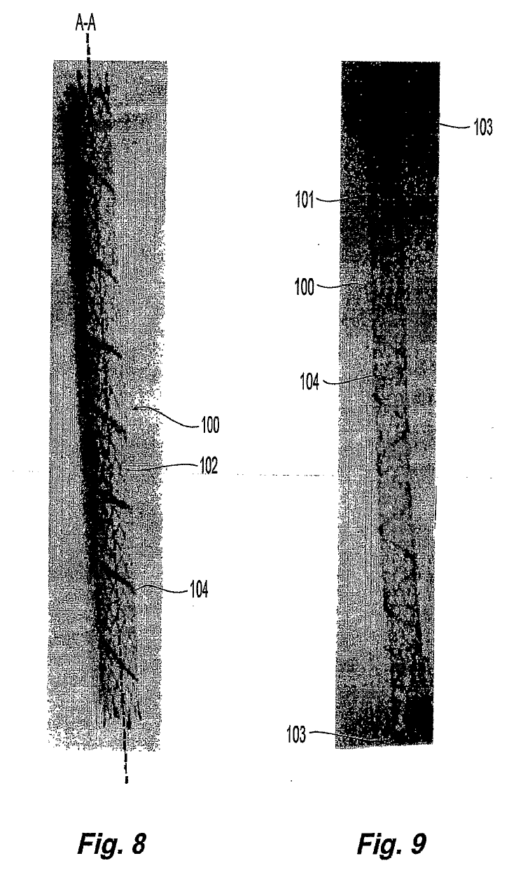

Shown in FIG. 8 is a preferred embodiment of an implantable prosthesis device, more preferably a stent graft 100 having an outer surface layer 102 and an inner layer (not shown) defining a central axis to engage, for example, a stenosis. The stent graft 100 and its outer surface 102 preferably define a substantially tubular member about the central axis A-A of the device 100. Preferably, the device 100 defines a substantially circular cross-section perpendicular to the central axis A-A, although other cross-sectional geometries are possible such as, for example, rectangular or oval. The device 100 is preferably configured for migration through a blood vessel to engage, for example, a stenosis.

The stent graft 100 preferably has a beading 104 to provide structural rigidity to the stent 100. More preferably, the beading 104 includes or is coupled to a radiopaque agent to form a radiopaque beading 104 to provide an operator with a visual location or orientation indicator during and following implantation of the implant 100 in a blood vessel. The radiopaque beading 104 is disposed within the stent graft 100 so as to substantially circumscribe the central axis. The radiopaque beading is preferably disposed between the inner and outer layers of the stent graft to define the contours of the device 100. The radiopaque beading 104 further preferably forms a continuous wrapping about the central axis of the device 100 so as to form a continuous contour line on the outer surface 102 of the device 100. Alternatively, the radiopaque beading 104 can be formed by a series of segments aligned about the outer surface 102. Further in the alternative, the radiopaque beading 104 can be formed by a plurality of individual rings dimensioned and configured to be disposed about the device 100 and spaced apart along the central axis. Each of the plurality of rings can define its own geometric shape, for example, a ring of beading may be substantially rectangular or circular so long as the ring defines a sufficient interstitial space to be disposed about the device 100.

The radiopaque beading 104 is preferably helically wound about the stent graft 100. The helical wrapping of the radiopaque beading 104 can maximize coverage of the surface 12 while minimizing the overall surface area of the beading 104 thereby minimizing the contact between the device 100 and any sheath used to install the device 100. The beading 104 preferably defines the line contact or contact surface of the device 100 when inserted in, for example, a delivery sheath. An exemplary delivery sheath includes FLUENCY® by Bard Peripheral Vascular, Tempe, Ariz. The minimized contact between the device 100 and the delivery sheath can minimize the force required to pull the sheath over device 100 during implantation. In addition, the preferred continuous helical wrapping beading 104 provides contour lines that provide additional visual cues to the user during and after implantation. For example, an untwisted implanted device 100 with preferred radiopaque beading 104 optimally appears as a series of parallel lines along the central axis of the device 100. Conversely, any twisting or bending in the device 100 would appear as converging lines in the radiopaque beading 104. Other coverage configurations for radiopaque beading 104 can be employed such as, for example, forming distinct circular radiopaque beading about the outer surface 102 along axial length of the device 100. The circular radiopaque beading 104 can be substantially perpendicular to the central axis or alternatively be oblique to the central axis. In another alternative coverage configuration, the beading 104 can be elongated strips of radiopaque beading radially spaced about the central axis of the device 100.

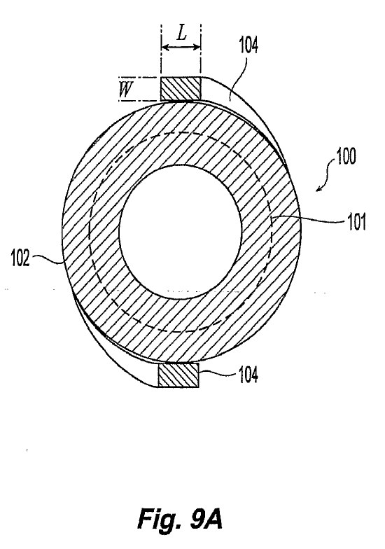

Referring to FIG. 9, a fluoroscopic or x-ray image of the device 100 of FIG. 8 is shown having the tantalum 60% and PTFE 40% beading 104 through a 15 millimeters aluminum plate. The plate is utilized to simulate the density of biological tissues by interposition of the plate (not shown) between the fluoroscope and the subject graft device. The radiopacity of the beading 104 is manifested in the imaging of the bead 104 contrasted with the radiolucent outer surface 102 of the device 100. Consequently, as long as an ordinary observer can determine that the lines provided by the radiopaque beading 104 in a fluoroscopic display medium, has a darker or higher contrast image than the remainder of the device 100, then the radiopacity of the beading 104 would be deemed to be greater than a minimum level needed for the beading to function as a radiopaque marker in a mammalian body. Alternatively, a machine vision with the ability to recognize discrete levels of contrast can be utilized to provide an objective indicator of the effectiveness of the radiopacity of the beading 104. The tantalum beading described above can also be visible to the naked unaided human eye.

Shown in FIG. 9A is a cross-sectional view of one embodiment of the device 100 with stent 101 encapsulated by inner and outer ePTFE material and the radiopaque beading 104. The radiopaque beading 104 is preferably rectangular in cross-section to provide the maximum contact surface for coupling to the device 10. Alternatively, the beading 104 can be any other geometry in cross-section such as, for example, circular, oval or polygonal. The preferred cross-sectional area of the beading 104 is dimensioned so as to have a length L of ranging from about 1 millimeter to about 2 millimeters and a width W ranging from about 100 microns to about 500 microns. More preferably, the cross-sectional area of the beading 104 is dimensioned so as to have a length L of about 1 millimeter and a width W of about 500 microns. Preferably, the elongated side of the beading 104 forms the interface between the radiopaque beading 104 and the exterior surface 102 of the device 100. In another preferred embodiment, the beading 104 is substantially circular in cross-section, and the diameter of the beading 14 is preferably about 0.67 millimeters. The radiopaque beading 104 can be formed by variety of techniques including extrusion, injection molding, solvent casting and the like.

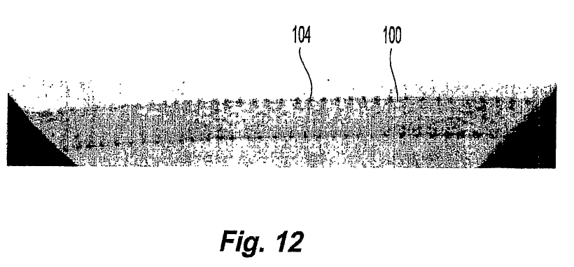

The radiopaque beading 104 can also be made of a biocompatible polyurethane material such as, for example, Carbothane PC-3575 by Noveon Inc. or other polymeric shell with a Barium Sulfate embedded in the polyurethane or polymeric shell as a radiopaque agent. The Carbothane material preferably has a 72 Shore D hardness and the Barium Sulfate is present at 20% by weight. Generally, a concentration of Barium Sulfate greater than 10% is sufficient to provide radiopacity. As shown in FIG. 12, a polyurethane beading with about 20% Barium Sulfate added is utilized in a spiral configuration about a stent-graft. Preferably, the concentration of Barium Sulfate in the beading 104 ranges from about 20% to about 40% to provide the radiopacity.

Referring back to FIG. 9A, the radiopaque beading 104 can be made from other biocompatible polymers, such as, for example, Dacron, polyester, PTFE, ePTFE, polycarbonates, polysulfone, polyethylene, polypropylene, polyurethane-urea, siloxane, and combinations thereof. In addition, other materials can serve as the radiopaque agents such as, for example, tantalum, tungsten, gold, silver or other metallic powders or salts such as calcium or HA salt.

The polymeric radiopaque beading 104 is preferably formed by extrusion. In one embodiment, the Carbothane PC-3575 material and about 20% by Barium Sulfate are combined in a composite resin or paste in which the Barium Sulfate is preferably substantially evenly dispersed throughout the polyurethane material. The composite paste is preferably loaded in a press device to compress the material into a billet. The billet is then preferably extruded to form the radiopaque polyurethane beading 104.

The polyurethane beading 104 is preferably solvent bonded to the PTFE surface. Although many methods of bonding such as sintering, heat melting can be used, a preferred bonding method involves the use of solvent for the beading material. For example, Carbothane FC-3575 is soluble in tetrahydrofuran (THF). THF is relatively low boiling solvent (boiling point <70° C.) and dissolves the polyurethane slowly. In a preferred method, a first layer of ePTFE encapsulation material (100 micron thick, 10-40 micron internal distance) is mounted on the steel mandrel and a stent is mounted on the ePTFE encapsulation layer. The radio-opaque polyurethane beading containing 20% Barium Sulfate is preferably spirally wound on the stent. Alternatively, other winding configurations can be used. Preferably, a second encapsulation membrane is mounted on the beaded stent. The entire assembly is dipped in a long measuring cylinder preferably containing 200 milliliters THF so as to expose all surface of the stent graft assembly to the THF. The assembly can be exposed to THF for 30 seconds to 5 minutes, more preferably to 1 minute. The exposure time is controlled so as to permit bonding of polyurethane beading to the ePTFE encapsulation layers without substantially dissolving the beading material. After the dipping, the beaded assembly can be taken out and air dried for 30 minutes and then dried in oven for 70° C. for 12 hours. If the ePTFE encapsulation material is sintered, no additional sintering step is needed, as the polyurethane beading holds the encapsulation layers together.

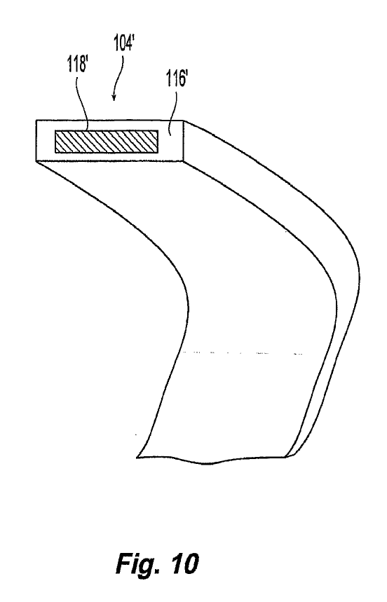

Another preferred embodiment of the radiopaque beading 104 is shown in FIG. 10 as radiopaque beading 104′ having an outer lumenal layer of non-radiopaque material 116′ surrounding a radioque core 118′. The outer layer 16′ is preferably ePTFE so as to provide an ePTFE beading 104′ with desired peeling properties in known beaded product such as CENTERFLEX® from Bard Peripheral Vascular, Tempe, Ariz. Alternatively, other polymeric materials can be used to form a shell to which the radiopaque agent can be coupled to or disposed within. Such polymeric materials include, for example, Dacron, polyester, polyurethane, PTFE, polycarbonates, polysulfone, polyethylene, polypropylene, polyurethane-urea, siloxane, and combinations thereof. The radiopaque core 118′ is preferably 20% by weight of Barium Sulfate salt material. Alternatively, the radiopaque core 118′ can be made from other radiopaque agents including tantalum, tungsten, gold, silver or other metallic powders or salts such as calcium or HA salt. The ePTFE beading 104′ can be made by a variety of suitable techniques, such as, for example, by extrusion of ePTFE and a suitable radiopaque material as described earlier to form a tubular extrudate or beading.

Alternatively, a radiopaque paste or resin can be partially or fully embedded in a portion of the inner surface of the PTFE resin without the use of an inner divider member 144. The radiopaque paste can be formed from a tantalum powder. For example, the radiopaque paste can be formed from a sixty-percent (60%) tantalum paste combined with an ePTFE paste. Additionally, other suitable materials can be utilized to form the radiopaque paste, for example, gold or titanium. Further in the alternative, the radiopaque paste can be formed from a Barium Sulfate mixture. For example, the radiopaque paste can be include art ePTFE paste mixed with about twenty to about forty percent (20-40%) Barium Sulfate. In a preferred embodiment, the radiopaque paste is formed into an elongated strip that can be disposed along the length of the inner surface of the PTFE resin. Alternatively or in addition to, the radiopaque paste can form a plurality of radiopaque elements that can be aligned along the inner surface of the PTFE resin along its length. The radiopaque paste can be formed into any shape or form. For example, the paste can be formed as sutures, threads and other small pieces such as disks disposed anywhere within the PTFE resin. A preferably continuous or elongated strip of radiopaque material embedded in the inner surface of the PTFE resin can provide the radiopaque core 118′ to the operator viewing the beading 104′ under fluoroscopy.

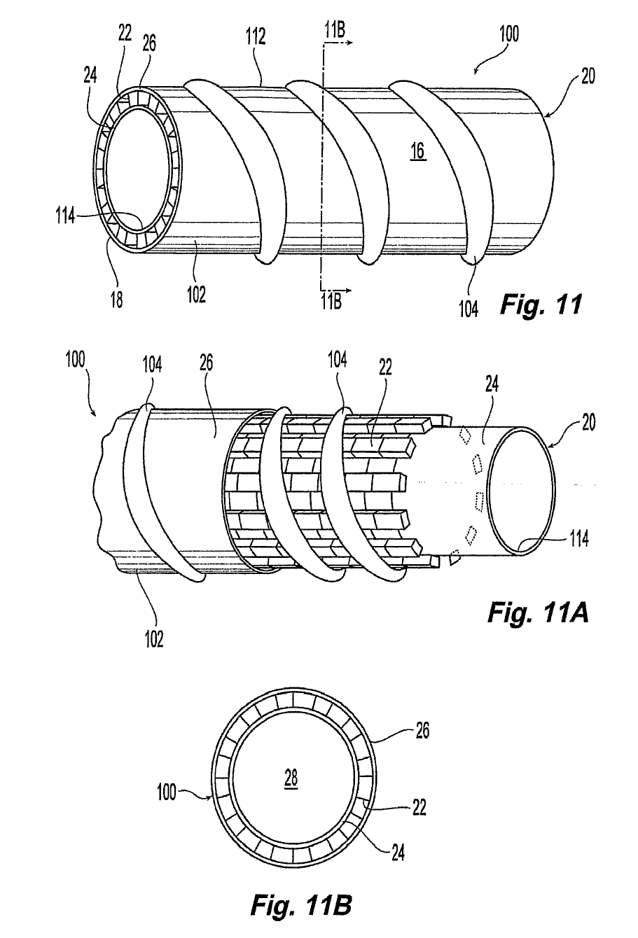

Referring to FIGS. 11, 11A, and 11B, the stent graft 100 can generally include a tubular member 112 having an interior surface 114 and an exterior surface 102 which are contained between first and second ends 18, 120. The tubular member 112 preferably includes a balloon or pressure expandable tubular shaped support frame or member 22 which is loaded over a first biocompatible flexible tubular member 24 that is held on a mandrel (not shown). A second biocompatible flexible tubular member 26 is then preferably loaded over the first biocompatible tubular member/support member combination 22, 24. The tubular shaped support member 22 preferably includes a stent similar to that described in U.S. Pat. Nos. 4,733,665; 6,053,941; 6,053,943; 5,707,386; 5,716,393, 5,860,999; and 6,572,647 each of which is incorporated in its entirety by reference. The stent utilized for the member 22 can be a balloon expandable stent, self-expanding stent or memory-shaped plastic stent. The tubular members 24, 26 are preferably fused together to encapsulate the support member 22.

The tubular members 24, 26 of stent-graph 100 are preferably formed by extruding a billet of expanded polytetrafluoroethylene (ePTFE). Alternatively, the first and second biocompatible flexible tubular members 24, 26 may also be made of unexpanded polytetrafluoroethylene (ePTFE). Further, the pressure expandable tubular shaped support member 22 may be made of any material having the strength and elasticity to permit radial expansion and resist radial collapse such as silver, titanium, stainless steel, gold, and any suitable plastic material capable of maintaining its shape and material properties at various sintering temperatures for PTFE or ePTFE. The tubular members 24, 26 can further alternatively be formed from other non-metallic materials such as, for example, Dacron, polyester, polyurethane, polyurethane-urea, siloxane, and combinations thereof. The material can include additional additives such as, for example, bio-active agents including hydroxyapatite (HA) to produce a material having increased biocompatibility and bioactivity. To form the tubular members 24, 26, the non-metallic material is preferably formulated into a resin or paste which is then compressed within a cylinder to form billet of the material, for example, an ePTFE billet. The billet is then preferably extruded and cured to form the tubular member. More preferably, the resin formulation, compression and extrusion techniques to form the tubular members 24, 26 are substantially similar to the techniques for forming the radiopaque beading described above.

Shown in FIG. 11B is a cross-sectional view of the stent graft 100 of FIG. 11 prior to fusing the graft or tubular members 24, 26 to the expansion member 22 to form the device 100. The first biocompatible flexible tubular member 24 forms the innermost layer or luminal surface of the stent graft 100, and further defines the lumen 28 of the stent graft 100, thereby providing a smooth, inert biocompatible blood flow surface. The tubular support member 22, preferably a stent, stent frame or similarly constructed structure, forms the middle layer located at the center of the stent graft 100. The second biocompatible flexible tubular member 26 forms the outermost layer or abluminal surface of the stent graft 100. To arrive at the stent graft device with radiopaque beading, the radiopaque beading is coupled or bonded to the stent graft device 100 at any suitable location. One location can be the outer surface of the inner member 24. Another location can be on the outer surface of the stent 22, as shown in FIG. 11A. Yet another location can be on the outer surface of the outer member 26. In each of these locations, the beading 104 is spirally wrapped about a longitudinal axis that extends through the device 100. Preferably, the beading 104 is wrapped and bonded to the outer surface of the device 100, as shown in FIG. 11.

Pressure is preferably applied to the graft/beading/stent/graft assembly in order to fuse the first and second biocompatible flexible tubular members 24, 26 to one another through the openings contained within the tubular support member 22 and between the spaces of the beading 104. Where the tubular support member 22 is a stent frame, the first and second ePTFE tubular members 24, 26 are fused in one another through the openings between the struts of the stent and between the spaces of winding of the beading 104.

The preferred techniques for fusing the radiopaque beading between the tubular members 24, 26 may vary depending upon the configuration and/or materials forming the radiopaque beading. For example, where the radiopaque beading is the polyurethane radiopaque beading 104 described above, the beading 104 is preferably dipped into an aprotic solvent (e.g., THF) and subsequently preloaded onto the outer surface of the inner tubular member 22 or alternatively outside the stent member 22. More specifically, the beading 104 is placed under tension, preferably about 500 grams of force as the beading 104 is wound onto the device 100. Alternatively, the entire graft/beading/stent/graft assembly can be sprayed by a coating of solvent that can dissolve polyurethane to form a mechanical bond between the beading 104 and the inner and outer layers of the assembly to form the stent graft device with radiopaque beading in FIG. 1. Where the beading is located between the stent frame 22 and the outer member 26, aprotic solvent can be sprayed onto the outer surface of member 26 such that the solvent migrates through the porous surfaces of member 26 to bond member 26 to the beading 104. Additionally, the entire assembly can be dipped into the aprotic solvent. Preferably, the solvent is tetrahydrofuran (THF), but other aprotic solvents can be used. The other solvents or solvent mixtures that can be used include acetone, dioxane, dimethyl acetamide, dimethyl sulfoxide, n-methyl pyrrolidinone and the like. Solvents or solvent mixtures with boiling points less than 100, more preferably less than 70° C. is most preferred. The solvent can be removed by preferably post-curing the assembled device 100 and beading 104.

In one embodiment, a 100 micron thick 6 millimeter diameter graft with 60% tantalum line (2 millimeter wide and 50-90 micron thick) was extruded. A tantalum filament can be produced by manually cutting the filament from the graft body using a razor blade. The filament is preferably used to produce radio-opaque markings on a graft or stent graft surface. The filament can be sintered or unsintered. An un-sintered filament is preferred because it can be fused with the graft body or stent graft body by sintering process. In another embodiment, a filament with 60% tantalum and 40% PTFE is spirally wound on an unsintered expanded graft surface. The filament and graft are preferably sintered to produce tantalum marking on the graft surface that is visible in x-ray imaging. The filament may also be spirally wound on a stent graft surface. In a preferred embodiment, the filament may be enclosed between two stent graft encapsulation layers.

In another preferred bonding technique, preferably for use where the radiopaque beading 14′ has an outer ePTFE shell and radiopaque core as described above, the graft/beading/stent/graft assembly is heated at sintering temperatures to form a physical bond between the layers. The sintering temperatures can range from about 100° C. to about 300° C., and preferably from about 100° C. to about 200° C.

The resulting prosthesis is an unexpanded stent and radiopaque beading encapsulated within ePTFE layers, or specifically, an unexpended stent having a radiopaque beading and ePTFE layers on its luminal and abluminal surfaces in which the stent, radiopaque beading and ePTFE layers are inseparable. Alternatively, the prosthesis can include hydroxyapatite on both its luminal and abluminal surfaces. Further, the ePTFE layers may also be fused or joined together around the ends of the unexpanded stent thereby entirely encasing the stent within ePTFE in both the radial and longitudinal directions. The resulting stent graft 100 and radiopaque beading can be loaded onto a suitable delivery device such as, for examples, U.S. Pat. No. 6,756,007, which is incorporated in its entirety by reference. The stent graft 100 may advantageously be used in a variety of medical applications including intravascular treatment of stenoses, aneurysms or fistulas; maintaining openings in the urinary, biliary, tracheobronchial, esophageal, renal tracts, vena cava filters; repairing abdominal aortic aneurysms; or repairing or shunting damaged or diseased organs such as, for example, Transjugular Intrahepatic Portosystemic Shunt (TIPS).

Referring back to FIG. 9, it is shown in this illustration a simulation of the density of biological tissues by interposition of the plate (not shown) between the fluoroscope and the subject graft device. The radiopacity of the beading 104 is manifested in the white or contrast imaging of the bead 104″ in comparison to the dark radiopaque “spoons” 103 at the ends of the device 100 and the lighter stent frame 101 of the device 100. Another example of a stent having “spoons” 103 is shown and described in U.S. Patent Application Publication No. 2004-0015228, which is incorporated in its entirety by reference. As long as an ordinary observer can determine that the lines provided by the radiopaque beading 104 in a fluoroscopic display medium has a contrasting image relative to, for example, the stent frame 101 or the spoons 103, then the radiopacity of the beading 104 would be deemed to be sufficient to function as a radiopaque marker in a mammalian body. Alternatively, a machine vision with the ability to recognize discrete levels of contrast can be utilized to provide an objective indicator of the effectiveness of the radiopacity of the radiopaque beading 104.

In yet another embodiment, a radiopaque beading in the form of a tape can be wrapped about a stent graft device 100. The cross-sectional area of such beading preferably is rectangular and is further preferably dimensioned such that the tape is about 2 millimeters wide with a thickness ranging from about 100 microns to about 150 microns. The tape can be formed with a preferred composite resin of about 60% tantalum as a radiopaque agent and 40% PTFE of polymeric material. Alternatively, other polymeric and radiopaque agents can be used. The tantalum and PTFE composite is preferably extruded and expanded three times to form the radiopaque tape. The tape is preferably bonded or coupled to a stent graft device 100 by wrapping the tape about a graft device 100 and sintering the assembly to fuse the radiopaque tape to the device 100.

In yet another embodiment, a hybrid stent-graft is provided in which the radiopaque material is co-extruded as part of the inner or outer members 24 or 26. Distinct from the prior embodiments of the stent-graft is the feature of the inner and outer members 24 and 26 encapsulating less than a major portion of the stent 22. That is, the stent graft of this embodiment has the appearance of about half of the stent being encapsulated by members 24 and 26, with about half of the stent being exposed or bare. The use of the radiopaque beading or tape in such hybrid stent-graft allows for a generally precise placement of the hybrid stent graft device in a procedure known as Transjugular Intrahepatic Portosystemic Shunt (TIPS) due to the ability of the clinician to view the extent of the covered portion of the stent via the radiopaque beading under fluoroscopic examination.

The design of the radiopaque beading allows applicant to achieve advantages that were previously unavailable. For example, the beading allows for lower loading and deployment forces because the contact surface is a continuous line rather than a cylinder. Second, the beading allows the graft or stent-graft to have increased kink resistance, i.e., a resistance to a change in the inside diameter of the graft or stent-graft as the prosthesis (graft or stent-graft) is curved about a small radius of curvature such as for example, 20 millimeters. Third, the spiral beading provides for an in-situ indication (via fluoroscopic imaging) of whether the graft or stent-graft has collapsed due to external pressure after implantation.

Finally, other types or bioactive agents can also be combined with the radiopaque materials described herein for the graft and the stent graft. The bioactive agents include (but are not limited to) pharmaceutic agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e., estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethaxone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents; vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

Although the stent graft device 100 has been described in relation to specific examples noted above, it should be emphasized that variations in the configuration or composition of ePTFE, radiopaque beading, stent framework, and other design parameters can be utilized with the graft device 100. Furthermore, the radiopaque beading provides additional visual cues to the operator beyond graft location.

As used herein, the singular form of “a”, “an,” and “the” include the plural referents unless specifically defined as only one. While the present invention has been disclosed with reference to certain preferred embodiments, numerous modifications, alterations, and changes to the described embodiments are possible without departing from the sphere and scope of the present invention, as defined in the appended claims. Moreover, where methods, processes and steps described above indicate that certain events occurring in certain order, those skilled in the art would recognize that the ordering of steps may be modified and that such modifications are within the variations of the described embodiments. Accordingly, it is intended that the present invention not be limited to the described embodiments, but that it have the full scope defined by the language of the following claims, and equivalents thereof.

We claim:

1. A graft device comprising:
a layer of synthetic non-metallic material having a first surface and a second surface spaced apart from the first surface;
a beading coupled to at least one of the first surface and the second surface of the layer; and
a radiopaque agent coupled to the beading to form a radiopaque beading.

2. The graft device according to claim 1, wherein the layer of synthetic non-metallic material forms an elongated substantially tubular member, the second surface forming the outer surface of the tubular member, and further wherein the radiopaque beading is spirally wrapped about the outer surface.

3. The graft device according to claim 1, wherein the radiopaque beading defines a substantially rectangular cross-sectional area.

4. The graft device according to claim 3, wherein the substantially rectangular cross-sectional area has a length ranging from about 1 millimeter to about 2 millimeters and a width ranging from about 100 microns to about 500 microns.

5. The graft device according to claim 4, wherein a side of the radiopaque beading defining the length of the cross-sectional area is coupled to at least one of the first surface and the second surface of the layer.

6. The graft device according to claim 1, wherein the radiopaque beading is tensioned and chemically bonded to the layer.

7. The graft device according to claim 1, wherein the radiopaque beading is sintered to the layer.

8. The graft device according to claim 1, wherein the radiopaque beading includes a radiopaque material embedded in a polyurethane material.

9. The graft device according to claim 1, wherein the radiopaque beading includes a radiopaque core disposed within a polytetrafluoroethylene shell.

10. The graft device according to claim 1, wherein the radiopaque material includes 20% by weight of Barium Sulfate.

11. The graft device according to claim 1, wherein the synthetic non-metallic material comprises a material selected from a group consisting essentially of Dacron, polyester, PTFE, ePTFE, polyurethane, polyurethane-urea, siloxane, and combinations thereof.

12. The graft device according to claim 1, wherein the radiopaque beading is formed from a paste having about 20% tantalum powder.

13. The graft device according to claim 1, wherein the radiopaque beading is formed from a paste having about 20% to about 40% Barium Sulfate.

14. The graft device according to claim 1, wherein the radiopaque beading is a tape comprised of about 40% tantalum powder and about 60% PTFE.

15. The graft device according to claim 1, wherein the radiopaque agent is at least partially embedded in the beading.

16. The graft device according to claim 1, wherein the beading comprises a continuous strip disposed helically about the device.

17. A method of forming a graft device comprising:
disposing a radiopaque agent in a polymeric shell;
compressing the radiopaque agent and shell to form a billet;
extruding the billet so as to form a radiopaque beading; and
wrapping the beading about a graft material so as to define a graft device.

18. The method of claim 17, wherein the wrapping includes preloading the beading about the graft.

19. The method according to claim 17, further comprising applying a solvent to at least one of the beading and the graft material.

20. A method of observing a position of a graft in a body, the method comprises:
disposing a graft having a radiopaque beading in a body;
exposing the body to an electromagnetic energy; and
fluoroscopically observing at least a portion of the beading to determine the position of the graft in the body.

21. A method of verifying orientation of a graft in a mammalian body subsequent to implantation of such graft in the mammalian body without an incision into the body, the method comprising:
directing electromagnetic energies at the implanted graft; and
forming an image on a display medium that shows the portion as a helically wound beading about the graft, the beading having greater contrast than another portion of the implanted graft.

22. An implantable prosthesis device comprising:
a stent frame having a first inner layer and a second outer layer defining a central axis; and
a beading coupled to at least one the layers.

23. The implantable prosthesis of claim 22, wherein the beading comprises a continuous strip disposed helically about the prosthesis.

24. The implantable prosthesis of claim 22, wherein the beading comprises a plurality of distinct segments disposed about the prosthesis.

25. The implantable prosthesis according to claim 22, wherein the beading is generally circumferentially disposed about the central axis.

26. The implantable prosthesis according to claim 22, wherein first and second layers are made of a synthetic non-metallic material.

27. The implantable prosthesis according to claim 26, wherein the synthetic non-metallic material of at least one of the layers comprises a material selected from a group consisting essentially of Dacron, polyester, PTFE, ePTFE, polyurethane, polyurethane-urea, siloxane, and combinations thereof.

28. The implantable prosthesis according to claim 22, wherein the beading is configured to be peeled.

29. The implantable prosthesis according to claim 22, further comprising a radiopaque agent coupled to the beading to form a radiopaque beading.

30. The implantable prosthesis according to claim 29, wherein the implantable prosthesis has first inner layer of synthetic non-metallic material and a second outer layer of non-metallic material spaced from the first layer, the radiopaque beading being disposed between the first and second layers.

31. The implantable prosthesis according to claim 29, wherein the radiopaque beading defines a substantially rectangular cross-sectional area.

32. The implantable prosthesis according to claim 31, wherein the substantially rectangular cross-sectional area has a length ranging from about 1 millimeter to about 2 millimeters and a width ranging from about 100 microns to about 500 microns.

33. The implantable prosthesis according to claim 31, wherein a side of the beading defining the length of the cross-sectional area is coupled to at least one of the layers.

34. The implantable prosthesis according to claim 29, wherein the radiopaque beading is coupled to the stent, tensioned and chemically bonded to at least one of the layers.

35. The implantable prosthesis according to claim 29, wherein the radiopaque beading is sintered to at least one of the layers.

36. The implantable prosthesis according to claim 29, wherein the radiopaque beading includes a radiopaque material embedded in a polyurethane material.

37. The implantable prosthesis according to claim 29, wherein the radiopaque beading includes a radiopaque core disposed within a polytetrafluoroethylene shell.

38. The implantable prosthesis according to claim 29, wherein the radiopaque beading includes 20% by weight of Barium Sulfate.

39. The implantable prosthesis according to claim 29, wherein the radiopaque beading is formed from a paste having about 20% tantalum powder.

40. The implantable prosthesis according to claim 29, wherein the radiopaque beading is formed from a paste having about 20% to about 40% Barium Sulfate.

41. The implantable prosthesis according to claim 29, wherein the radiopaque beading is a tape of 40% tantalum powder and 60% PTFE.

42. A method of forming an implantable prosthesis device comprising:
disposing a radiopaque agent in a polymeric shell;
compressing the radiopaque agent and shell to form a billet;
extruding the billet so as to form a radiopaque beading; and
wrapping the beading about a graft material so as to define an implantable prosthesis device.

43. The method of claim 42, wherein the wrapping includes preloading the beading about the implantable prosthesis.

44. The method of claim 42, further comprising applying a solvent to at least one of the beading and graft.

45. A method of observing a position of a graft comprises:
disposing a graft on a body;
observing the portion of the beading on the body surface.

46. A method of verifying orientation of a implantable prosthesis in a mammalian body subsequent to implantation of such implantable prosthesis in the mammalian body without an incision into the body, the method comprising:
directing electromagnetic energies at the implanted implantable prosthesis;
blocking some of electromagnetic energies through a portion of the implantable prosthesis; and
forming an image on a display medium that shows the portion as a helically wound beading about the implantable prosthesis, the beading having greater contrast than an ePTFE material.

47. A method of observing a position of a implantable prosthesis in a body, the method comprises:
disposing a implantable prosthesis having a radiopaque beading in the body;
exposing the body to an electromagnetic energy; and
fluoroscopically observing at least a portion of the beading to determine the position of the implantable prosthesis in the body.

48. A method of forming a beading for a vascular graft comprising:
combining a radiopaque agent and a polymeric resin to form a composite;
extruding the composite so as to form a radiopaque beading.

49. The method of claim 48 further comprising forming the composite into a billet.

50. The method according to claim 48, further comprising expanding the beading to form a tape.
